(19) United States
(12) Patent Application Publication
(10) Pub. No.: US 2003/0109858 A1
(43) Pub. Date: Jun. 12, 2003

(54) Disposable surgical instrument

(76) Inventor: Tomoaki Koseki, Tokyo (JP)

Correspondence Address: Tomoaki Koseki, 7-3 Komagome 7 Chome, Toshima-ku Tokyo, 170 0003, JP

(21) Appl. No.: 10/182,750

(22) PCT Filed: Nov. 28, 2001
(86) PCT No.: PCT/JP01/10358

(30) Foreign Application Priority Data
Dec. 4, 2000 (JP) 2000-403754

(51) Int. Cl. A61B 17/00

(52) U.S. Cl. 606/1

(57) ABSTRACT

Affordable, easy-to-use, and labor-saving disposable surgical instruments, in which the entire form or the grip part of the instruments molded by heat press processing method, after having the metal part of the instruments as the core enfolded with foaming material such as polyethylene foam or polyurethane foam.

### BACKGROUND OF THE INVENTION

1. Field of the Invention

This invention relates to medical instruments utilized in surgical operations.

2. Description of the Prior Art

Generally, as entire surgical instruments are made of metal, nurses use a specific detergent exclusive for washing metal to wash off blood and other dirt once the surgery is over. The washed surgical instruments are then fully dried, then coating agent such as oil is applied, kept per category on a specific shelf or a container exclusive to each instrument item, and the instruments are sterilized with auto-clave sterilization before the next surgery. In cases of ethylene oxide gas sterilization, the instruments are sealed in a sterilization bag and then sterilized. Material for parts of surgical instruments are usually made with cut-shave process method with lathe, milling machine, and wire cutters, the parts are then welded or combined to make the whole instrument.

### SUMMARY OF THIS INVENTION

The process of washing, re-sterilization and re-use of surgical instruments is not necessarily economically efficient when including nurses' labor cost, expense of consumables such as washing detergents, coating agents, ethylene oxide gas, and also the overall maintenance cost of sterilization equipments and the total management expense.

Furthermore, in areas of plastic surgery and such, it is now a common practice in the industry for instrument manufacturers and import agents to lend the instruments free of charge under the condition that the companies' consumable products be purchased by the medical organization, and so instrument manufacturers or import agents attend surgical operations, the manufacturers or agents collect the lent out instruments after the operation and then conduct maintenance management of the instruments on behalf of medical organizations. The management expense is naturally added onto the price of the consumables, the proper insurance redemption price is distorted, and this practice is becoming a large cause in creating price difference between overseas market and Japanese domestic market. Furthermore, the manufacturers and agents are forced to bear the burden that is originally to be borne by the medical staff, and this practice is becoming an obstacle against healthy development of these companies, losing track of proper efforts to provide better products and services in the medical field. Nurses attending operations are not necessarily exclusive nurses for surgeries, so they would often rotate within hospital wards, out-patient areas and other areas in the hospital. News of medical accidents are reported frequently in recent years, and one of the elementary mistakes of an inexperienced nurse is infection.

Although medical instrument manufacturers are imposed by the Ministry of Health and Welfare of even stricter GMP of sterilized medical instruments and stricter standard for sterilization validity, these standards and GMP are still not applied to medical organizations. Washing and rinsing, and re-sterilization of surgical instruments do require certain know-how and experience. Also, even for well-experienced nurses, washing and rinsing center parts of cylindrical instruments and very narrow deep aperture parts, is very difficult. Incomplete handling of the instruments is directly linked to infection accidents.

There are many cases where the production volume lot of each surgical instrument item is less than 10, and the number of items with orders of more than 100 is limited. Therefore it is difficult to manufacture products with press processing manufacturing method or casting method, methods more suitable for mass production. When instruments are manufactured with only metal material, the cost becomes high, as the large concave convex parts in grips and such parts have to be shaved. Unless expensive roulette process and the like are conducted on the grip parts, instruments easily slip with mere continuance of horizontal and vertical ditches. As for hook-shaped parts of intestinal spatulas and brain spatulas, the metal parts touch organs directly, and damage the organs. Therefore, nurses wind and wrap the tip ends of those instruments with pieces of gauze to alleviate the invasiveness in each operation.

There are some instruments currently used, of which the entire instrument is made of plastic resin, but the usage is limited to simple treatments in hospital wards, and not much used in operation rooms. Plastic cannot be the substitute for metal, as the intensity and the spring characteristic, flexibility and other features of plastic are not suitable as a material for instruments to be used in an operation room to operate conveying a surgeon's delicate manual feelings. Also, an injection molding process for manufacturing of resin made instruments requires very precise mold shaping, and the high mold cost is a burden expense wise.

To solve the above problems, this invention utilizes polyethylene foam, polyurethane foam and other 25 foaming materials, having the metal part of the surgical instruments to be the core, the surrounding is wrapped and enfolded, then heat is applied on top from a molding device, compressed, melt and adhered, heat press processing is applied, thus the grip part or the entire instrument is shaped and formed with this manufacturing method, and the surgical instrument is made as a disposable instrument to be used only once.

### DESCRIPTION OF THE PREFERRED EMBODIMENTS

Hereafter, a description of the preferred embodiments of this invention is made in reference to drawings. FIG. 1 shows a device which measures the depth of a meniscus plate, the grip 1 of which is formed of heat press processing method, a method in which polyethylene foam foaming material according to claim 1 and 3 is utilized, having the metal shaft 2 as the core, the foaming material enfolding around the metal shaft core, then pressed, melted and adhered with heat after shaping. The upper diagram is a cross-sectional diagram, which shows the latter part of the inserted shaft 2 with projection 3 that is bent 90 degrees upward to prevent rotation and loosening. The surface of grip 1 produces moderately rough feeling and is slip proof when cloth is applied on the shaped foam. The surface of the foam has cushion characteristics in comparison to metal surface, and fits one's hands comfortably. When letters are engraved on the shaped foam, the letters become visible as embossed letters 4 on the surface of grip 1, and therefore it is easier to distinguish each instrument from one another. Also as air bubbles inside the polyethylene foam are connected, the foam has high moisture absorption ability and thus absorbs blood well. Therefore this feature ensures that the instrument will be used only once as a disposable instrument, preventing re-usage due to misidentification. The metallic characteristic such as intensity and springy characteristics of shaft 2 are maintained, and the shaft 2 having the same diameter from the insert end to the tip part can be finished affordably as it only requires bending process using a press machine.

FIG. 2 shows an intestinal spatula, the entire instrument of which is enfolded and shaped with polyethylene foam 7a, and 7b according to claim 2 and 3. All the periphery is surrounded with heat-melt adhered part 6. When using this instrument, the middle part is bent as in the lower diagram. Conventionally, metal part 5 is wrapped with pieces of gauze to prevent invasion onto organs, but nurses' labor is decreased as the whole instrument is wrapped with polyethylene. As pieces of gauze are not used, naturally it is unnecessary to count the gauze pieces. As all the metal part is covered, aluminum and other such easy-to-process affordable material can be utilized.

This invention has the following effects, conducted as in the above description. The processing of the metal part is simplified, by utilizing foaming material such as polyethylene, polyurethane, and by enfolding the surrounding part of the instrument having the metal as the core, also by having the grip part of the whole instrument shaped and formed with heat press processing, and as this instrument is utilized only once, the usage of the instrument increases, and enables manufacturing with heat press processing or a casting method suitable for mass production at a low cost. The heat press processing for shaping is more affordable, and allows smaller manufacturing lot compared to injection molding method.

The grip surface made of polyethylene foam has higher slip-proof effect, has higher cushion characteristic compared to metal surface, and fits one's hands comfortably. Letters can be engraved and are visible as embossed letters, and so each instrument can be distinguished easily. This grip part has high moisture absorption ability, and absorbs blood well. Because of this feature, this instrument will certainly be used only once as a disposable instrument, and this feature prevents any re-usage due to misidentification.

The entire intestinal spatula is wrapped with polyethylene foam, reducing nurses' labor. As no pieces of gauze are used, there is no need for gauze piece counting. As all the metal part is covered, low cost and easy-to-process material such as aluminum can be used.

As the instruments are disposable, this invention facilitates the reduction of nurses' labor of re-sterilization work, facilitates cost reduction of instrument management, and leads to prevention of infection accidents.

As the instruments are disposable, this invention slows the free-of-charge lending action conducted by instrument manufacturers and import agents. Appropriate insurance redemption price of consumables will be realized, and this will shrink the price difference between overseas market and domestic market. Manufacturers and import agents can concentrate on their primary work.

### BRIEF DESCRIPTION OF THE DRAWINGS

FIG. 1 is a cross-section and external drawing of an instrument that measures the depth of meniscus plate.

FIG. 2 shows external drawings of an intestinal spatula, one in a state when half of the melt adhered part is pulled up, and the other of a state when the spatula is bent for actual usage.

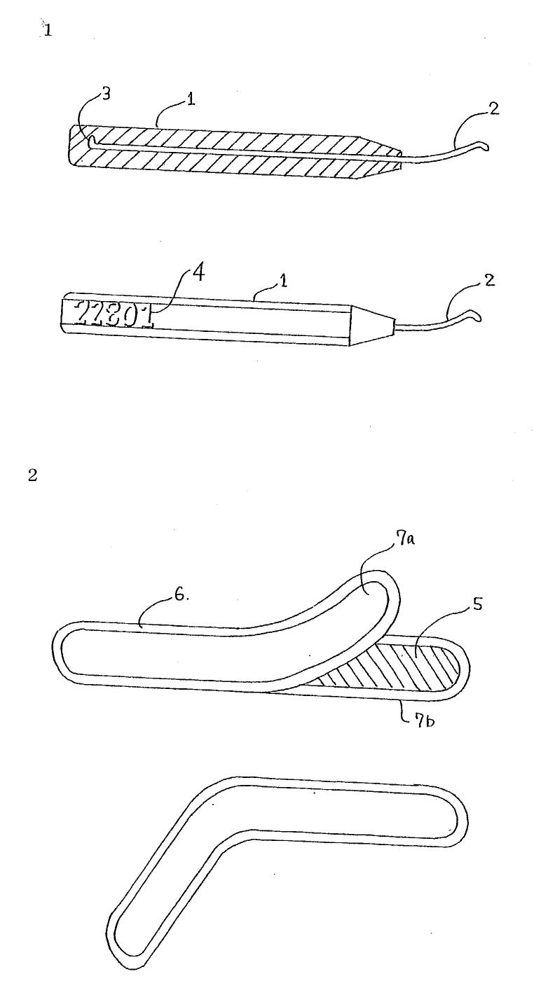

What is claimed is:

1. Disposable surgical instruments with the characteristic of being disposable to be used only once, instruments comprising hooks, probes, driving devices, extractors, chisels, hammers, rasps, scalpels, suction tubes, needle-holders, tweezers, forceps, knives, and such, in which the grip part to hold the instrument is shaped by being enfolded and wrapped with foaming material such as polyethylene or polyurethane foam.

2. The disposable surgical instruments according to claim 1, for instruments such as hooks which directly touch the organs, having the characteristic of enfolding and wrapping not only the grip part but the entire instrument to the tip end utilizing the above foaming material, to alleviate the invasiveness onto organs.

3. The manufacturing method of the disposable surgical instruments according to claim 1 and claim 2, utilizing polyethylene, polyurethane and other such foaming material, to wrap and enfold the surrounding of the instrument, having the metal part as the core, apply heat press processing method, in which heat is applied on top of the enfolded instrument, and shaped and form after pressing, melting and adherence is applied, to shape the grip part or the whole of the instruments.
